(19) United States
(12) Patent Application Publication
(10) Pub. No.: US 2025/0228904 A1
(43) Pub. Date: Jul. 17, 2025

(54) SYNBIOTIC COMPOSITIONS FOR METABOLIC MANAGEMENT ESPECIALLY GLUCOSE METABOLISM MANAGEMENT AND MODULATION OF SATIETY HORMONE LEVELS

(71) Applicant: Evonik Operations GmbH, Essen (DE)

(72) Inventors: Heike tom Dieck, Friedrichsdorf (DE); Bodo Speckmann, Kahl (DE)

(73) Assignee: Evonik Operations GmbH, Essen (DE)

(21) Appl. No.: 18/702,912

(22) PCT Filed: Jun. 23, 2022
(86) PCT No.: PCT/EP2022/067100

§ 371 Date: Apr. 19, 2024

(30) Foreign Application Priority Data
Oct. 21, 2021 (EP) 21203871.5

(51) Int. Cl. A61K 35/742; A61K 9/50; A61K 36/82; A61K 36/9066; A61K 38/05; A61P 3/04; A61P 3/10

(52) U.S. Cl. CPC A61K 35/742; A61K 9/5026; A61K 36/82; A61K 36/9066; A61K 38/05; A61P 3/04; A61P 3/10

(57) ABSTRACT

A synbiotic preparation contains at least one probiotic strain and at least one amino acid or derivative displaying an effect in glucose metabolism management, such as diabetes prevention or prediabetes treatment. The preparation modulates satiety hormones, such as CCK, GLP-1, and PYY. Methods are developed to treat obesity, adiposity, type 2 diabetes, and metabolic syndrome with the preparation. A preparation also decreases fasting glucose levels in a subject.

The current invention concerns a synbiotic preparation for use in the modulation of satiety hormone levels in a subject, wherein the preparation comprises at least one probiotic Bacillus subtilis strain, and at least one dipeptide comprising a glutamine or glutamic acid unit, and wherein the satiety hormone is selected from CCK, GLP-1 and PYY. Moreover, this preparation displays an effect in glucose metabolism management e.g. diabetes prevention or prediabetes treatment/reversion to healthy.

The gut microbiota is a crucial modulator of health effects elicited by food components. The microbial metabolite butyrate has emerged as an important and targetable mediator of such effects, whereas a relative butyrate deficiency has been associated with several intestinal and metabolic diseases. Available prebiotic strategies for stimulation of butyrate production in the human gut, such as the application of FODMAPs, can cause unwanted side-effects, e.g. diarrhea, abdominal pain, and flatulence, especially in people with food intolerances or irritable bowel syndrome, and consequently have limited applicability. We therefore aimed to develop a FODMAP-free, synbiotic composition as novel means of shifting both microbiota composition and activity towards butyrate production.

The gastrointestinal microbiota forms an intriguing and interconnected relationship with orally ingested matter—be it food or pharmaceutical ingredients—and human physiology. In that sense, microbiota composition and activity are affected by diet, and on the other hand dietary molecules are converted through a plethora of (microbe-specific) metabolic pathways to a partly absorbable metabolome. Examples of gut microbiota-derived metabolites with known effects on the host include phenolic acids, indole derivatives, and short-chain fatty acids (SCFA) acetate, propionate, and butyrate. Butyrate is an important energy source and differentiation factor for colonic epithelial cells, it also supports the formation of mucin as well as tight junction proteins and thereby contributes to intestinal barrier integrity [1]. Furthermore, butyrate can trigger anti-inflammatory signaling via binding to arylhydrocarbon, GPR41, GPR109, and PPARγ receptors and has consequently been related to the etiology of inflammatory bowel diseases (IBD), which display reduced levels of butyrate and of butyrate-producing bacteria [1].

A fraction of around two percent of luminal butyrate enters the circulation [2] via the portal vein, and it thereby also affects tissues beyond the gastrointestinal tract, such as the liver, adipose tissue, and pancreas. The systemic actions of a sufficient butyrate supply can be summarized as a (beneficial) modulation of cardio-metabolic health (glucose and lipid metabolism leading to increased insulin sensitivity, reduced plasma glucose and cholesterol levels, as well as increased satiety and decreased blood pressure [3]). Although available studies have not established a causal role for butyrate or other SCFA in the pathophysiology of metabolic disorders, it appears likely that they at least partly contribute to the health effects caused by e.g. fibre-rich diets [4, 5]. In conclusion, an increase of colonic butyrate levels is an attractive target for the development of microbiota-targeted intervention strategies to achieve targeted health outcomes, in particular for the prevention and treatment of IBD and metabolic disorders such as type 2 diabetes, traits of the metabolic syndrome, and cardiovascular diseases. Especially of interest would be to manage borderline diabetes (Condition of Prediabetes) to prevent development of full-blown diabetes, as this is kind of reversible. With development of full diabetes (definition) a reversion becomes much more difficult.

Prediabetes is when the blood sugar level is higher than it should be but not high enough for a diagnose of diabetes. They might call it impaired fasting glucose or impaired glucose tolerance. People with type 2 diabetes almost always had prediabetes first, which usually doesn't cause symptoms. A significant percentage of people especially in western countries have prediabetes, but 90% don't know that they have it. Prediabetes treatment can prevent more serious health problems, including type 2 diabetes and problems with heart, blood vessels, eyes, and kidneys.

Colonic butyrate levels can be targeted by means of prebiotics, probiotics, combinations thereof and also by direct intake of butyric acid in the form of salts or butyrate precursors, such as tributyrin. Sodium butyrate has been clinically assessed as a co-treatment of IBD patients, e.g. supporting the efficacy of 5-ASA in refractory distal ulcerative colitis in a topical application [6]. However, convenient and patient-friendly application forms are crucial compliance factors, even more so in a preventative approach, hence oral interventions are favorable. For butyrate this has been realized by a delayed-release coated tablet formulation targeting butyrate to the distant small intestine/colon [7]. The kinetic profile indicated a sudden intraluminal release of butyrate from the carrier, which supposedly is disadvantageous over a more sustained uptake of butyrate formed continuously within the gastrointestinal tract. Moreover, the intense odor of butyrate-containing supplements limits their applicability.

Probably the most well-established means of triggering this formation is the ingestion of prebiotic fibers, either through the normal diet or via fortified foods or dietary supplements. The most investigated and applied prebiotics are fructo-oligosaccharides (FOS), galacto-oligosaccharides (GOS), arabinoxylan-oligosaccharides (AXOS), xylo-oligosaccharides (XOS), and beta-glucans. These carbohydrates form part of the larger group of FODMAPs (fermentable oligosaccharides, disaccharides, monosaccharides, and polyols). Diets low in FODMAP content have increasingly become popular [8], as they often cause unwanted side-effects, e.g. diarrhea, constipation, and flatulence. A low FODMAP diet effectively reduces these symptoms in patients with irritable bowel syndrome [9]. The use of fibers as source of intestinal SCFA is therefore of limited applicability at least for certain populations. An alternative approach is the direct application of butyrate-producing bacteria as supplemental or therapeutic probiotics. The human gut microbiota is known to use four catabolic pathways that lead to butyrate [10], with three of them processing proteins/amino acids as educts, and the pyruvate/acetyl-CoA pathway (Ac pathway) processing proteins as well as carbohydrates, the latter being the most abundant in a multi-ethnic metagenome analysis [11]. Faecalibacterium prausnitzii, Oscillibacter, and Clostridium XIVa, comprise the dominant core community of all bacteria associated with the Ac pathway. The species of these taxa do not belong to the commonly used probiotic bacteria of the genera Lactobacillus, Bifidobacterium, and Bacillus, but have more recently been studied and applied as so-called next-generation probiotics, e.g. Akkermansia muciniphila, Faecalibacterium prausnitzii, Clostridium butyricum, Clostridium beijerinckii, and Eubacterium hallii. Though these developments are clearly interesting, a recent pilot trial assessing a cocktail including four of the above-mentioned next-generation probiotic strains failed to achieve a significant induction of faecal butyrate levels [12]. Another Clostridium butyricum strain has been characterized as a butyrate-producer in vitro and in rodents, but its butyrate-producing capacity in the human gut remains to be established [13]. Likewise, a 4-week intervention with 10⁸ CFU/day of a Butyricicoccus pullicaecorum strain had no effect on faecal butyrate [14]. Overall, the application of next-generation/butyrate producing probiotics in humans is intriguing but also facing technical challenges, due to their strict anaerobic nature, making them difficult to produce and ensure long-term stability in formulated finished products.

A nutritional product comprising Bacillus subtilis DSM 32315 and alanyl-glutamine in a colon-targeted capsule formulation leading to SCFA production is disclosed in EP3784807A1.

For the present invention, in a clinical pilot trial involving 18 healthy volunteers was performed and it was shown that the formulation can safely improve butyrate levels in humans. Four-week supplementation was well tolerated and significantly increased fecal butyrate levels, accompanied by changes in microbiota. Moreover, circulating lipid parameters were significantly improved, GLP-1 and PYY was significantly reduced an influence on fasting blood glucose were detected leading to a reduction of subjects having a prediabetic fasting blood glucose level to zero after 4 weeks of supplementation compared to 3 prediabetic subjects before start.

Moreover and surprisingly, the improvement of blood glucose metabolism as shown by reduction of number of prediabetic people after intervention was accompanied by a significant reduction of GLP-1 and PYY levels. Normally, an (postprandial) increase of the satiety hormones would be expected [15]. It might be a marker of improvement of metabolic health comparable to that of weight loss, as this is one of the very rare conditions for which a reduction of total circulating satiety hormones is described in connection to a positive effect [16, 17]. The significant reduction accompanying the supplementation can be rated as a marker for improvement. One hypothesis for the observed reduction of fasting GLP-1 could be that GLP-1 secretion may be inhibited by circulating non-esterified fatty acids as reported by Ranganath et al.[18]. In the present case, Bacillus subtilis together with the substrate dipeptide (Alanyl-Glutamin) could have increased the production of SCFAs especially in the upper gut parts, supporting the hypothesis. Only few studies focus on fasting plasma concentrations of PYY and GLP-1. One of those is the study of Luis et al. [16]. In an obese study collective, a significant decrease of basal GLP-1 levels was observed in subjects with weight loss after a hypocaloric diet. This was accompanied with a significant improvement in anthropometric parameters and cardiovascular risk factors, e.g. reduction of LDL-cholesterol, triglycerides, Insulin and HOMA-index. Furthermore, a significant correlation was seen in the study by Luis et al. between basal insulin and GLP-1 levels. Fasting insulin was not measured in the current study, however, a slight decrease in glucose levels was also seen in the normoglycaemic group. The reduction of GLP-1 concentration after weight loss is also reported by others [17], but there are also publications showing an increase of GLP-1 concentrations after weight loss [19]. Therefore, different mechanisms seem to be responsible and there might be also differences during weight loss and during weight maintenance. Regardless, we here disclose for the first time a distinct GLP-1 and PYY reduction in a normal weight study collective in response to a symbiotic dietary supplement.

The lipid status, especially the blood biomarkers total cholesterol and LDL cholesterol showed a significant decrease throughout the study. This is in line with previous in vivo studies which showed that the administration of e.g. Lactobacillus probiotics is effective in improving lipid profiles, including the reduction of total cholesterol and LDL cholesterol [20]. These cholesterol-lowering effects can be partially ascribed to bile salt hydrolase activity (BSH) activity. Deconjugated bile salts are less efficiently reabsorbed than their conjugated counterparts, which results in the excretion of larger amounts of free bile acids in feces. Also, free bile salts are less efficient in the solubilization and absorption of lipids in the gut. Therefore, deconjugation of bile salts could lead to a reduction in serum cholesterol either by increasing the demand for cholesterol for de novo synthesis of bile acids to replace those lost in feces or by reducing cholesterol solubility and thereby absorption of cholesterol through the intestinal lumen. Additional modes of action of probiotics are also reported.

Collinsella, especially Collinsella aerofaciens might be of further interest for health applications in the future. Recent studies identified butyrate-producing species in the genus of Collinsella [21]. In addition Collinsella is described to be beneficial in several further publications, such as in WO2010125421A, describing the use of Collinsella aerofaciens for reducing bloating as well as in WO2016038198A1 disclosing the use of Collinsella for treatment of inflammatory bowel disease.

In conclusion the intervention indicates that this synbiotic composition provides an effective and safe tool for stimulation of intestinal butyrate production with subsequent positive effects on the modulation of satiety hormone levels.

The present invention is therefore directed to a preparation for use in the modulation of satiety hormone levels in a subject, wherein the preparation comprises
at least one probiotic Bacillus subtilis strain, and
at least one dipeptide comprising a glutamine or glutamic acid unit, and
wherein the satiety hormone is selected from CCK, GLP-1 and PYY.

In a preferred configuration, the preparation further comprises one or more plant extracts selected from a curcuma extract and green tea extract.

In a preferred configuration, the preparation is to be administered to the subject at least 1 oral dosage of at least 1 billion CFU of the probiotic Bacillus subtilis strain and at least 250 mg dipeptide per day, preferably at least 2 oral dosages per day.

In a specific configuration, the modulation of satiety hormones is a reduction of satiety hormone level of at least 5% or at least 10% or at least 20%, or at least 30% after 4 weeks of administration of the preparation.

In a specific configuration, the dosage is at least 1 billion CFU of the probiotic Bacillus subtilis strain and at least 250 mg dipeptide per day.

In the fasting state, however, people with normal glucose tolerance had significantly lower GLP-1 plasma concentrations than people with Type 2 Diabetes [24,25]. In a case-control study, GLP-1 levels were positively correlated with metabolic syndrome traits [25], whereas fasting GLP-1 was reduced in obese subjects following weight-loss diets [26,27].

Such a reduction of (potentially elevated) satiety hormone level—as a potential sign of beginning metabolic syndrome—is beneficial, as this shows a re-balancing of the metabolic status, such as seen with a weight loss diet [26,27].

It is further preferred, when the preparation is administered in the form of capsules or tablets, comprising an enteric coating.

According to the present invention, it is preferred when the probiotic strain is selected from Bacillus subtilis DSM 32315, Bacillus subtilis DSM 32540, Bacillus subtilis DSM 32592, preferably Bacillus subtilis DSM 32315.

The dipeptide is preferably selected from Glycine-Glutamine, Glycine-Glutamic acid, Alanine-Glutamine, Alanine-Glutamic acid and its acetylated forms.

In a specific configuration, the dipeptide is L-Alanyl-L-Glutamine.

In a preferred embodiment, the total amount of probiotic strain and amino acid or oligopeptide is at least 40 weight-%, preferably at least 50 weight-% more preferably at least 60 weight-%, most preferably at least 70 weight-% of the total weight of the preparation.

It is further preferred, when the total amount of plant extracts is at least 10 weight-%, preferably at least 20 weight-% more preferably between 20 and 40 weight-% of the total weight of the preparation.

In another preferred embodiment, the preparation comprises an enteric coating, wherein the enteric coating comprises one or more of the following: methyl acrylate-methacrylic acid copolymers, cellulose acetate phthalate (CAP), cellulose acetate succinate, Hydroxypropyl methyl cellulose phthalate, hydroxypropyl methyl cellulose acetate succinate (hypromellose acetate succinate), polyvinyl acetate phthalate (PVAP), methyl methacrylate-methacrylic acid copolymers, shellac, cellulose acetate trimellitate, sodium alginate, zein, preferably a methyl acrylate-methacrylic acid copolymer.

In another preferred embodiment, the preparation comprises an enteric coating comprising a polymer composition, wherein the polymer is polymerized from 20 to 30% by weight methyl methacrylate, 60 to 70% by weight methyl acrylate and 8 to 12% by weight methacrylic acid. It is further preferred, when the polymer is polymerized from 25% by weight methyl methacrylate, 65% by weight methyl acrylate and 10% by weight methacrylic acid.

In another aspect of the present invention, the preparation according to the present invention is for use in the prevention and treatment of obesity, adiposity, type 2 diabetes, metabolic syndrome.

Another aspect of the present invention is related to a preparation for use in the decrease in fasting glucose in a subject, preferably below a fasting glucose value of 100 mg/dl, wherein the preparation comprises
at least one probiotic Bacillus subtilis strain, and
at least one dipeptide comprising a glutamine or glutamic acid unit.

In a preferred configuration, the probiotic Bacillus subtilis strain is Bacillus subtilis DSM 32315 and the dipeptide is Alanine-Glutamine, preferably L-Alanyl-L-Glutamine.

In a specific configuration, fasting glucose is reduced after 4 weeks of administration of at least 1 oral dosage of at least 1 billion CFU of the probiotic Bacillus subtilis strain and at least 250 mg dipeptide per day, preferably at least 2 oral dosages per day.

In another specific configuration, the glycemic response in the subject is reduced, preferably in response to a glucose test meal.

The glycemic response can be determined 2 hours after a standardized glucose test meal.

In a specific configuration, the subject is human and in a prediabetic state, having a fasting glucose value of more than 100 mg/dl.

A fasting blood sugar level of 99 mg/dL or lower is normal, 100 to 125 mg/dL indicates that the subject has prediabetes, and 126 mg/dL or higher indicates that the subject has diabetes.

Fasting glucose value can be determined as the baseline glucose level, which is predicted on daily individual 24-hours glucose profiles as well as data from the medical anamnesis.

Another possibility to determine, if a subject is in a prediabetic state is the determination of the level of glycated hemoglobin in % (HbA1c), where a subject is considered prediabetic, having a HbA1c level of more than 5.7%.

In a specific configuration, the glycemic response is reduced after 4 weeks of administration of at least 1 oral dosage of at least 1 billion CFU of the probiotic Bacillus subtilis strain and at least 250 mg dipeptide per day, preferably at least 2 oral dosages per day.

In a specific configuration of the present invention, the body weight of the subject is reduced by at least 1 kg after 4 weeks of administration of the preparation. This reduction of body weight is independent of calorie intake and physical activity of the subject.

Therefore, the preparation is administered to the subject at least 1 oral dosage of at least 1 billion CFU of the probiotic Bacillus subtilis strain and at least 250 mg dipeptide per day, preferably at least 2 oral dosages per day.

### WORKING EXAMPLES

### Human Study I

The study was performed in an open-label design with a 2-weeks lasting run-in period (basal characterization of subjects with regard to occurrence of gastrointestinal symptoms and bowel function), followed by a 4-weeks lasting intervention period. After informed consent, healthy subjects were screened for their eligibility to take part in the study. At the beginning, after 14 days and at the end of intervention (28 days) stool samples and blood samples were collected for biomarker determination and microbiome analysis. To control for confounding factors, the diet was recorded with a food frequency protocol 3 days prior to each visit. Blood routine parameters were determined at each visit after at least 10 hours overnight fast.

The synbiotic product composition in HPMC capsule with colonic coating (daily dose=2 capsules) was:

Bacillus subtilis DSM 32315 spore powder (˜with 2 billion CFU), L-Alanyl-L-Glutamine (290 mg), 90 mg extract of Curcuma, 90 mg extract of green tea, D- and B-vitamin(s) and mineral per capsule.

The capsule comprises a colonic coating with EUDRAGUARD® biotic, which can start to disintegrate at pH conditions in the lower small intestine and the colon (pH>7.0). The coating comprises a polymer composition, wherein the polymer is polymerized from 20 to 30% by weight methyl methacrylate, 60 to 70% by weight methyl acrylate and 8 to 12% by weight methacrylic acid. It is further preferred, when the polymer is polymerized from 25% by weight methyl methacrylate, 65% by weight methyl acrylate and 10% by weight methacrylic acid.

### Intake of the Investigational Product

The product composition as described above was taken as one capsule in the morning (flexible with or without breakfast) and one capsule in the evening (flexible with or without dinner) unchewed with water. The study day prior to study visits, the intake of the evening capsule was standardized and taken 12±2 hours prior to the scheduled visit at study site.

The following parameters were determined:

Fasting glucose was determined (as well as differentiated haemogram, liver enzymes (GPT, GOT, γ-GT, AP), creatinine, uric acid).

Routine parameters were determined at the routine lab. Analysis of blood routine parameters/differentiated haemogram was performed within 24 hours after blood sampling.

Blood samples for routine parameters and haemogram as well as lipid profile were shipped the same day to blood routine lab.

Blood routine parameters were checked at screening, visit 1, 2 and 3. Blood sampling was performed at study after at least 10 hours overnight fast. Preparation: Clotting for 30 min at room temperature, 10 minutes centrifugation at 3000×g and 4° C. Blood samples for routine parameters and haemogram as well as lipid profile were shipped the same day to blood routine lab.

Lipid status (triglycerides, total cholesterol, HDL- and LDL-cholesterol) were determined in serum the same day after each visit at the routine lab. Total cholesterol, HDL-cholesterol and triglycerides were determined photometrically. LDL-cholesterol was calculated according to Friedewald calculation.

For the assessment of total GLP-1 and PYY, DPP-IV and AEBSF inhibitor was added to the EDTA-plasma tube prior to blood collection. The prepared tubes were stored frozen until blood collection.

Total GLP-1 and PYY were analysed in plasma at BioTeSys GmbH using ELISA kits (Merck Millipore EZGLP1T-36K for total GLP-1 and EZHPYYT66K for PYY).

For GLP-1 and PYY analysis, samples were aliquoted and stored below −70° C. until shipment or analysis.

### Example 1: Influence of 4-Week Supplementation of Synbiotic Product on Number of Prediabetic Subjects

TABLE 1
Categories of fasting blood glucose levels; frequency [n]

| Fasting blood glucose [mg/dL] | Baseline | 2 weeks | 4 weeks |
|---|---|---|---|
| ≥100 mg/dL (prediabetic) | 3 | 1 | 0 |
| ≥90 mg/dL to <100 mg/dL | 7 | 8 | 10 |
| ≥80 mg/dL to <90 mg/dL | 8 | 9 | 7 |
| ≥70 mg/dL to <80 mg/dL | 0 | 0 | 1 |
| Total | 18 | 18 | 18 |

Three subjects had elevated/prediabetic fasting blood glucose levels at baseline. At the end of intervention after 4 weeks, all subjects had fasting blood glucose levels within (healthy) reference range (see Table 1).

During the course of the study for glucose a reduction was observed over the study period (see Table 2).

TABLE 2
Descriptive statistics of fasting blood glucose levels [mg/dl]

| Glucose [mg/dL] | Baseline | 2 weeks | 4 weeks |
|---|---|---|---|
| Number of values | 18 | 18 | 18 |
| Minimum | 82.0 | 85.0 | 73.0 |
| 25% Percentile | 86.8 | 87.0 | 87.8 |
| Median | 93.0 | 90.0 | 91.0 |
| 75% Percentile | 95.5 | 94.3 | 93.0 |
| Maximum | 106.0 | 101.0 | 98.0 |
| Mean | 92.06 | 90.94 | 89.50 |
| Std. Deviation | 6.66 | 4.49 | 5.51 |
| Std. Error | 1.57 | 1.06 | 1.30 |
| Lower 95% CI of mean | 88.74 | 88.71 | 86.76 |
| Upper 95% CI of mean | 95.37 | 93.18 | 92.24 |

Example 2: Influence of 4-Week Supplementation on GLP-1 and PYY Level Accompanied by Metabolic Improvement (Cholesterol Ad Glucose) not Directly Correlated to Significant Changes in Butyrate Level

Moreover, the gastrointestinal hormones total GLP-1 and PYY were measured in fasting blood samples at each visit. Both hormones showed a significant decrease between baseline and end of intervention. Gastrointestinal hormones play an important role in the communication between the gastrointestinal tract and the brain, mediating signals of hunger and satiety. GLP-1 and PYY are anorexigenic hormones that are secreted by the gastrointestinal tract into the circulation in response to a meal, reducing appetite and food intake. PYY and GLP-1 play important roles in the regulation of food intake and insulin secretion, and are of translational interest in the field of obesity and diabetes. PYY production is highest in enteroendocrine cells located in the distal intestine, mirroring the sites where high concentrations of short chain fatty acids (SCFAs) are produced by gut microbiota (Larraufie et al. 2018). During the study, mean levels of total GLP-1 decreased significantly from 23.11 pmol/L to 14.89 pmol/L. In addition, mean PYY levels decreased from 96.44 pg/mL to 57.52 pg/mL between baseline and end of intervention after 4 weeks.

One hypothesis for the observed reduction of fasting GLP-1 could be that GLP-1 secretion may be inhibited by circulating non-esterified fatty acids as reported by [18]. In the present case, Bacillus subtilis together with the substrate dipeptide (Alanyl-Glutamine) could have increased the production of SCFAs especially in the upper gut parts, supporting the hypothesis. Only few studies focus on fasting plasma concentrations of PYY and GLP-1. One of those is the study of Luis et al. [16]. In an obese study collective, a significant decrease of basal GLP-1 levels was observed in subjects with weight loss after a hypocaloric diet. This was accompanied with a significant improvement in anthropometric parameters and cardiovascular risk factors, e.g. reduction of LDL-cholesterol, triglycerides, Insulin and HOMA-index. Furthermore, a significant correlation was seen in the study by Luis et al. between basal insulin and GLP-1 levels. Fasting insulin was not measured in the current study, however, a slight decrease in glucose levels was also seen in the normoglycaemic group. The reduction of GLP-1 concentration after weight loss is also reported by others [17], but there are also publications showing an increase of GLP-1 concentrations after weight loss [19]. Therefore, different mechanisms seem to be responsible and there might be also differences during weight loss and during weight maintenance. Regardless, the interesting findings with distinct GLP-1 and PYY reduction in the normal weight study collective in response to SAMANA® FORCE should be further investigated. As a next step we propose to evaluate also the impact on postprandial secretion of GLP-1 and PYY levels after a meal.

### GLP-1

GLP-1 is a gut hormone for appetite control released from endocrine cells in the gut. Fasting blood levels were measured at baseline (V1), after 2 weeks of intervention (V2) and at the end of intervention after 4 weeks (V3). Total GLP-1 levels decreased significantly between baseline and end of intervention after 4 weeks (p<0.001). Already after 2 weeks of intervention, total GLP-1 levels decreased significantly (p<0.001) (see Table 3).

TABLE 3
Descriptive statistics of total GLP-1 levels [pmol/L]

| GLP-1 [pmol/L] | Baseline | 2 weeks | 4 weeks |
|---|---|---|---|
| Number of values | 18 | 18 | 18 |
| Minimum | 11.1 | 4.6 | 5.2 |
| 25% Percentile | 18.5 | 8.9 | 10.0 |
| Median | 22.5 | 14.6 | 13.7 |
| 75% Percentile | 27.1 | 17.5 | 18.3 |
| Maximum | 41.0 | 23.6 | 30.2 |
| Mean | 23.11 | 14.09 | 14.89 |
| Std. Deviation | 7.60 | 5.63 | 7.09 |
| Std. Error | 1.79 | 1.33 | 1.67 |
| Lower 95% CI of mean | 19.33 | 11.29 | 11.37 |
| Upper 95% CI of mean | 26.88 | 16.89 | 18.42 |

### PYY

PYY is an anorexigenic gut hormone for appetite control. In line with total GLP-1, PYY levels decreased significantly between baseline and end of intervention after 4 weeks (p<0.001). Already after 2 weeks of intervention, PYY levels decreased significantly (p<0.0139) (see Table 4).

TABLE 4
Descriptive statistics of PYY levels [pg/mL]

| PYY [pg/mL] | Baseline | 2 weeks | 4 weeks |
|---|---|---|---|
| Number of values | 18 | 18 | 18 |
| Minimum | 39.8 | 25.5 | 13.3 |
| 25% Percentile | 72.1 | 47.0 | 37.9 |
| Median | 96.5 | 77.1 | 54.0 |
| 75% Percentile | 118.4 | 112.0 | 79.2 |
| Maximum | 184.6 | 129.0 | 100.4 |
| Mean | 96.44 | 78.04 | 57.52 |
| Std. Deviation | 34.01 | 33.39 | 25.98 |
| Std. Error | 8.02 | 7.87 | 6.12 |
| Lower 95% CI of mean | 79.53 | 61.43 | 44.60 |
| Upper 95% CI of mean | 113.40 | 94.65 | 70.44 |

### Example 3: Influence of 4-Week Supplementation on Lipid Parameter

The lipid status, especially the blood biomarkers total cholesterol and LDL cholesterol showed a significant decrease throughout the study.

All blood samples were collected under fasting conditions. Total cholesterol, LDL cholesterol, HDL cholesterol as well as triglycerides were determined. The majority of the subjects showed levels within reference range (see dotted line). For total cholesterol and triglycerides, fasting blood reference values are indicated with <190 mg/dL and <150 mg/dL, respectively. For HDL cholesterol, heathy blood levels are indicated with >40 mg/dL.

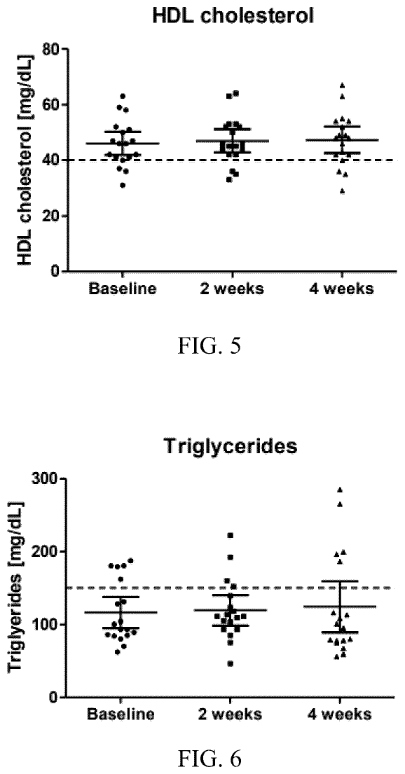

Total cholesterol levels as well as LDL cholesterol levels decreased significantly between baseline and end of intervention after 4 weeks (see FIG. 4 and FIG. 5 and Tables 5 and 6). On average, HDL cholesterol levels slightly increased from 46.06 mg/dL to 47.33 mg/dL throughout the study, but not significantly (see FIG. 5 and Table 6).

Moreover, triglycerides levels did not change significantly over the study period (see FIG. 6 and Table 7).

TABLE 5
Descriptive statistics of total cholesterol levels [mg/dL]

| Total cholesterol [mg/dL] | Baseline | 2 weeks | 4 weeks |
|---|---|---|---|
| Number of values | 18 | 18 | 18 |
| Minimum | 126.0 | 112.0 | 119.0 |
| 25% Percentile | 162.3 | 158.8 | 155.0 |
| Median | 178.0 | 174.0 | 170.0 |
| 75% Percentile | 192.3 | 189.3 | 185.8 |
| Maximum | 240.0 | 225.0 | 228.0 |
| Mean | 179.30 | 172.40 | 169.10 |
| Std. Deviation | 29.87 | 30.04 | 27.27 |
| Std. Error | 7.04 | 7.08 | 6.43 |
| Lower 95% CI of mean | 164.50 | 157.50 | 155.50 |
| Upper 95% CI of mean | 194.20 | 187.40 | 182.70 |

TABLE 6
Descriptive statistics of HDL cholesterol levels [mg/dL]

| HDL cholesterol [mg/dL] | Baseline | 2 weeks | 4 weeks |
|---|---|---|---|
| Number of values | 18 | 18 | 18 |
| Minimum | 31.0 | 33.0 | 29.0 |
| 25% Percentile | 40.8 | 42.0 | 41.5 |
| Median | 46.0 | 45.5 | 48.0 |
| 75% Percentile | 51.3 | 52.3 | 54.0 |
| Maximum | 63.0 | 64.0 | 67.0 |
| Mean | 46.06 | 46.94 | 47.33 |
| Std. Deviation | 8.42 | 8.45 | 9.57 |
| Std. Error | 1.98 | 1.99 | 2.26 |
| Lower 95% CI of mean | 41.87 | 42.74 | 42.58 |
| Upper 95% CI of mean | 50.24 | 51.15 | 52.09 |

TABLE 7
Descriptive statistics of triglyceride levels [mg/dL]

| Triglycerides [mg/dL] | Baseline | 2 weeks | 4 weeks |
|---|---|---|---|
| Number of values | 18 | 18 | 18 |
| Minimum | 62.0 | 46.0 | 56.0 |
| 25% Percentile | 84.8 | 93.0 | 77.3 |
| Median | 96.5 | 111.0 | 98.0 |
| 75% Percentile | 166.3 | 142.3 | 188.5 |
| Maximum | 187.0 | 222.0 | 285.0 |
| Mean | 116.30 | 119.40 | 124.20 |
| Std. Deviation | 42.76 | 41.73 | 70.69 |
| Std. Error | 10.08 | 9.84 | 16.66 |
| Lower 95% CI of mean | 95.01 | 98.69 | 89.07 |
| Upper 95% CI of mean | 137.50 | 140.20 | 159.40 |

TABLE 8
Descriptive statistics of LDL/HDL cholesterol ratio

| LDL/HDL cholestero ratio | Baseline | 2 weeks | 4 weeks |
|---|---|---|---|
| Number of values | 18 | 18 | 18 |
| Minimum | 1.2 | 1.0 | 0.9 |
| 25% Percentile | 2.0 | 1.9 | 1.7 |
| Median | 2.6 | 2.6 | 2.6 |
| 75% Percentile | 3.5 | 3.0 | 3.2 |
| Maximum | 4.8 | 5.2 | 4.1 |
| Mean | 2.73 | 2.62 | 2.47 |
| Std. Deviation | 1.02 | 1.10 | 0.89 |
| Std. Error | 0.24 | 0.26 | 0.21 |
| Lower 95% CI of mean | 2.22 | 2.08 | 2.02 |
| Upper 95% CI of mean | 3.23 | 3.17 | 2.91 |

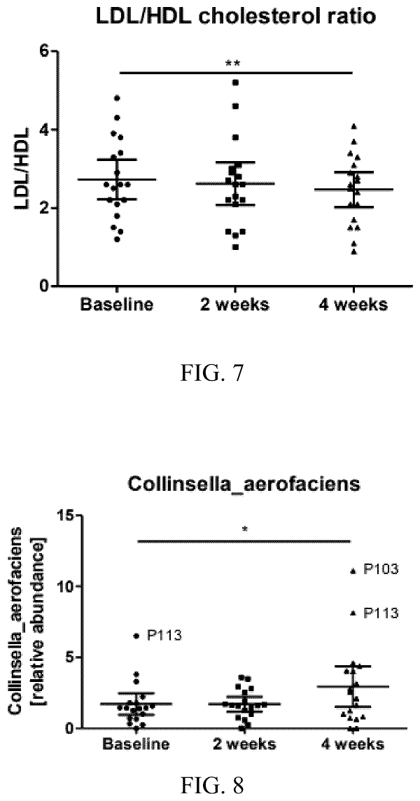

In literature, the LDL/HDL cholesterol ratio is often calculated to estimate the risk for atherosclerosis and coronary heart disease. The ideal ratio is indicated with <3.0, with higher values increasing the risk of heart disease [22]. In the present study, LDL/HDL cholesterol ratio decreased significantly between baseline and end of intervention after 4 weeks (see FIG. 7 and Table 8).

### Example 4: Increase in Abundance of Collinsella aerofaciens in Fecal Samples

An increase of the phylum Actinobacteria was confirmed on class level. This increase can be ascribed to both the order of Coriobacteriales and Bifidobacteriales as well as the family of Coriobacteriaceae and Bifidobacteriaceae. Confirming the findings on order level of Coriobacteriales and on family level of Coriobacteriaceae, a significant increase was also observed in the genus Collinsella. The significant change in the genus Collinsella can be ascribed to the species Collinsella aerofaciens (see FIG. 8). Surprisingly, this increase in Collinsella by the combination of B. subtilis and Alanyl-Glutamine was detected in in-vitro gut flora and gut model, as well as in this human study (see FIG. 8).

### Human Study II

### Course of the Study

At the beginning of the study, participants applied the MillionFriends program (www.millionfriends.de) provided by Perfood GmbH. Therefore, continuous tissue glucose monitoring (Abbott Freestyle libre system) was performed for 14 days, including 1 day of sensor calibration. The baseline values of the parameters of interest were measured within these 14 days. This included the blood glucose reactions of the participants to prescribed and standardised test meals as well as other parameters to describe the blood glucose. Participants also answered questions about their digestion and their well-being in general. In addition, the participants took a stool sample and sent it in for analysis.

Following the first sensor assisted sensor-phase, the participants began to consume SAMANA® FORCE. They were advised to take 2 unchewed capsules daily. They were free to decide at what time they took the capsules and whether they wanted to take them with or without a meal. Apart from that, the participants continued their diet as usual.

After two weeks in which the patients regularly consumed SAMANA® FORCE and filled in questionnaires, followed the second sensor-assisted testphase. Once again, continuous tissue glucose monitoring (Abbott Freestyle libre system) was performed for 14 days, including 1 day of sensor calibration. The intake of SAMANA® FORCE was continued during the second sensor phase. Glycemic responses to the test meals and parameters describing blood glucose were measured again. In addition, a second stool sample was taken and the questionnaires about digestion and well-being were answered again.

Only after the second sensor phase was completed did the participants receive a nutrition report containing personalised dietary recommendations based on the blood glucose responses during the first sensor phase.

The synbiotic product composition in HPMC capsule with colonic coating (daily dose=2 capsules) was:

Bacillus subtilis DSM 32315 spore powder (˜with 2 billion CFU), L-Alanyl-L-Glutamine (290 mg), 90 mg extract of Curcuma, 90 mg extract of green tea, D- and B-vitamin(s) and mineral per capsule.

The capsule comprises a colonic coating with EUDRAGUARD® biotic, which can start to disintegrate at pH conditions in the lower small intestine and the colon (pH>7.0). The coating comprises a polymer composition, wherein the polymer is polymerized from 20 to 30% by weight methyl methacrylate, 60 to 70% by weight methyl acrylate and 8 to 12% by weight methacrylic acid. It is further preferred, when the polymer is polymerized from 25% by weight methyl methacrylate, 65% by weight methyl acrylate and 10% by weight methacrylic acid.

### Standardized Glucose Meal Test

The participants were asked to drink a standardized glucose test meal in both sensor-assisted test phases. For this, they received to weighed packets of glucose (60 g) with their test kit. Participants should dissolve the packet in 200-300 ml water and drink it in the morning on an empty stomach at the beginning of each sensor phase.

### Determination of Fasting Glucose, HbA1c and Glycemic Responses

Fasting glucose was determined as the baseline glucose level using an in-house developed, proprietary algorithm. Briefly, the baseline is predicted on daily individual 24-hours glucose profiles as well as data from the medical anamnesis. HbA1c (in %) was calculated by multiplying the average glucose level of the complete test phase with 0.03 and adding 2.6. Of note, HbA1c as calculated in this study is not of the same quality as HbA1c measured in standardized laboratories.

To analyze glycemic responses to test meals, AUCi (incremental area under the curve) was determined for 120 minutes after the logged meal intakes. The trapezoidal rule was applied in the calculation. Participants were in-structed to have a minimum distance of 2 hours between 2 meals and/or a meal and a physical activity.

During the first 2 weeks subjects followed their blood sugar levels by minimal invasive continuous blood sugar measurement (reading out via a mobile application), detecting the blood sugar reaction on their daily meals and nutrition, which was documented in a diary. In addition, they performed 3 challenges with standard meals (glucose (adapted glucose tolerance test), white bread with protein and fat spread and whole grain bread). This phase was followed by a 4 week-intervention phase ingesting the symbiotic product (daily) for 14 days without blood sugar measurement and the following 14 days with the same minimal invasive continuous blood sugar measurement (reading it out via a mobile application), detecting the blood sugar reaction on their daily meals and nutrition in a diary. They performed 3 challenges with standard meals (glucose (adapted glucose tolerance test), white bread with protein and fat spread and whole grain bread) again. A stool sample for microbiota analysis was taken before and after the study.

### Example 5: Data on Weight Loss

Results showed a significant weight loss independent of nutritional adaptions (as the eating pattern shown by unchanged macronutrient supply and distribution remained the same) improved blood sugar reaction in the area under the curve in the adapted glucose tolerance test.

Via social media canals 192 study participants were recruited. The study cohort was in 65.1% female (n=125) and 34.9% male (n=67). Mean age was 42.86 (±12.48) years. Mean weight was 80.28 kg (±17.88) with a mean BMI of 26.88 kg/m² (±6.17).

Neither the energy nor the average amount of protein and fat per day differed significantly between both sensor phases. Regarding physical activity, participants were less active during the second sensor phase (p<0.001).

Subgroup with Prediabetes

A closer look at the blood glucose parameters measured within the first sensor phase revealed that several participants had higher levels of fasting glucose and HbA1c values. In 99 participants the calculated Hba1c was above 5.7% while the fasting glucose of 62 participants was above 100 mg/dl. Since a fasting glucose above 100 mg/dl and HbA1c values above 5.7% indicate the presence of prediabetes, participants could be divided into a prediabetic (n=62) and non-prediabetic (n=118) subgroup based on these two criteria.

These two groups were analysed separately. Therefore, the eating behaviour and level of physical activity of participants with or without should also be analysed separately as well, in order to identify these factors as possible confounders within these groups.

62 study participants were considered prediabetic. These participants were in 53.23% female (n=33) and 46.03% male (n=29). Mean age was 42.52 (±12.18) years. Mean weight was 84.18 kg (±16.20) with a mean BMI of 27.60 kg/m2 (±6.45).

Neither the energy nor the average amount of carbohydrates, protein, fat and fibers per day differed significantly between both sensor phases. Regarding physical activity, participants with prediabetes were less active during the second sensor phase (p=0.016).

Subgroup without Prediabetes

118 study participants did not have signs of a prediabetes. These participants were in 69.49% female (n=82) and 30.51% male (n=36). Mean age was 41.16 (±12.10) years. Mean weight was 78.13 kg (±18.35) with a mean BMI of 26.45 kg/m2 (±5.98).

Regarding the intake of macronutrients, no significant difference could be found in the average amount of fat and protein intake per day or the daily intake of kilocalories. However, the intake of carbohydrates (p=0.001) and fibers (p<0.001) was reduced in the second sensor phase. Regarding physical activity, participants without prediabetes were less active during the second sensor phase (p<0.001).

### Comparison of Body Weight and Composition

Besides a possible change in glycemic responses, changes in body weight were also of interest. The participants were asked to report their current weight and waist-to-hip ratio (whr) at the beginning and end of the study. For this purpose, a measuring tape was provided to the participants in the test kit. Only participants whose fasting glucose was known and who reported their weight or WHR at both time points were included in the analysis.

The comparison of body weight at the beginning and end of the study showed a significant weight loss of participants (p<0.001). Significant weight loss has also been shown for prediabetics (p<0.001) and non-prediabetics (p<0.001).

In addition to body weight, it was also examined whether there were any changes in waist-to-hip ratio. In contrast to the body weight, no significant changes could be observed in WHR. This applied to the analysis of both the study population and the subgroups of prediabetics and non-prediabetics.

The following table shows a summary of the analyses regarding body weight and composition (see table 9).

TABLE 9
Comparison of body weight before and after the
regular intake of SAMANA ® FORCE

| Variable | n | before | | after | | p-value |
|---|---|---|---|---|---|---|
| Body weight | 171 | 80.71 | (±18.00) | 79.64 | (±17.43) | <0.001¹ |
| (kg) | | | | | | |
| Prediabetes | 58 | 84.67 | (±16.53) | 83.21 | (±15.94) | <0.001¹ |
| No Prediabetes | 113 | 78.68 | (±18.44) | 77.81 | (±17.93) | <0.001¹ |
| BMI (m/kg²) | 171 | 26.85 | (±5.65) | 26.50 | (±5.47) | <0.001¹ |
| Prediabetes | 58 | 27.28 | (±4.86) | 26.80 | (±4.61) | <0.001¹ |
| No Prediabetes | 113 | 26.63 | (±6.03) | 26.35 | (±5.88) | <0.001¹ |
| Waist-to-hip- | 171 | 0.87 | (±0.10) | 0.87 | (±0.09) | 0.621 ² |
| ratio | | | | | | |
| Prediabetes | 58 | 0.89 | (±0.11) | 0.89 | (±0.10) | 0.510 ² |
| No Prediabetes | 113 | 0.86 | (±0.09) | 0.86 | (±0.09) | 0.923 ² |

¹Wilcoxon-test
² paired t-test

The absolute reduction of body weight throughout the study course in summarized in table 10.

TABLE 10
Absolute reduction of body weight

| Group of persons | Δ weight in kg |
|---|---|
| Whole study group | −1.07 |
| Prediabetes | −1.46 |
| No Prediabetes | 0.87 |

### Analysis of Fasting Glucose

The fasting glucose in the first and second sensor phase were compared to answer the question if the intake of SAMANA® FORCE influences fasting glucose (n=180). Fasting glucose was determined as the baseline glucose level using an in-house developed, proprietary algorithm. Briefly, the baseline is predicted on daily individual 24-hours glucose profiles as well as data from the medical anamnesis.

The results showed a significant reduction of fasting glucose with supplementation of SAMANA® FORCE (p<0.001) and are summarized in table 11.

TABLE 11
Comparison of fasting glucose before and after
supplementation of SAMANA ® FORCE

| Variable | n | before | after | Diff. | p-value |
|---|---|---|---|---|---|
| fasting glucose | 180 | 96.92 (±8.29) | 94.58 (±9.27) | −2.34 | <0.001* |
| (mg/dl) | | | | | |

*paired t-test

### Subanalysis of Prediabetic Participants

The previous results raised the question of whether fasting glucose was reduced in all participants or whether a certain group of participants carried the effect. Therefore, it was checked whether there were people among the participants whose fasting glucose at the beginning of the intervention indicated the presence of prediabetes (>100 mg/dl).

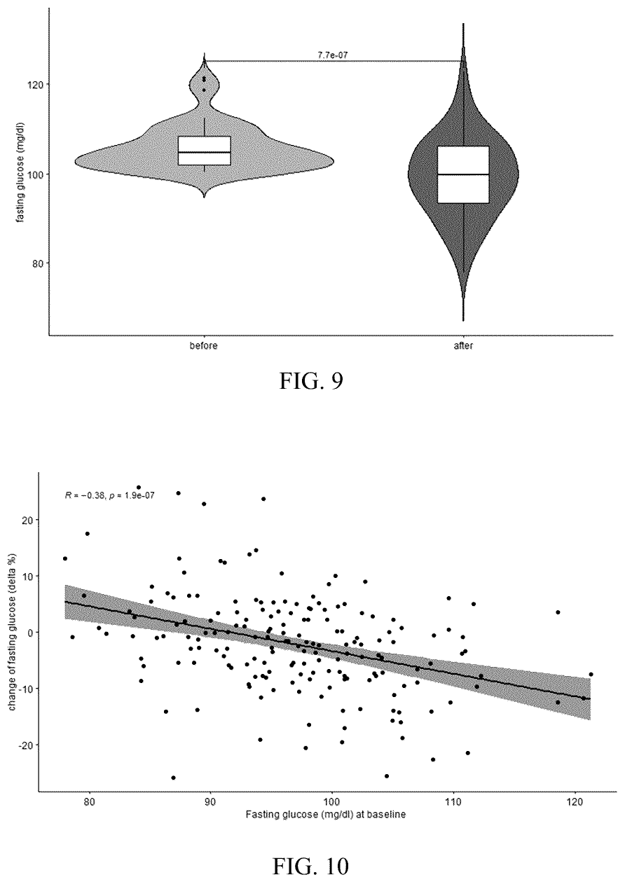

At first, the data of participants with (n=62) and without prediabetes (n=118) were analysed separately. A closer look at participants with prediabetes confirmed a significant decrease in fasting glucose after regular intake of SAMANA® FORCE (p<0.001) and is shown in FIG. 9.

33 people fell below a fasting glucose value of 100 mg/dl and were therefore no longer counted in the prediabetic category at the end of the study.

In contrast, no significant change in fasting glucose in participants without prediabetes was found (p=0.582).

Additionally, the percentage changes (delta %) in fasting glucose over the course of the study were compared between participants with and without prediabetes. In line with the previous results, there was a significantly greater decrease in fasting glucose in the prediabetic group (p<0.001). The results are summarized in table 12.

TABLE 12
Comparison of fasting glucose level (in mg/dl) before and
after of supplementation of SAMANA ® FORCE in
participants with and without prediabetes (>100 mg/dl glucose).

| Variable | n | before | after | p-value | delta (%) |
|---|---|---|---|---|---|
| Prediabetes | 62 | 105.73 | 99.23 | <0.001* | −6.10 |
| | | (±5.09) | (±9.12) | | (±7.89) |
| No Prediabetes | 118 | 92.29 | 92.14 | 0.582* | −0.04 |
| | | (±5.37) | (±8.39) | | (±8.65) |

*Wilcoxon Test

These results suggest that the possible effects of SAMANA® FORCE on fasting glucose might depend on the level of the baseline fasting glucose. To get to the bottom of this assumption, a correlation analysis was carried out. According to the analysis, there was a significant correlation between the level of fasting glucose at baseline and the percentage change of fasting glucose (R=−0.38, p<0.001). The higher the initial value, the greater the decrease in fasting glucose. The analysis is shown in FIG. 10.

Analysis of Glycemic Response after Glucose Test Meal

To address the question whether the daily intake of SAMANA® FORCE led to differences in postprandial glycemic responses, the 2 hours responses after a standardized glucose test meal were compared.

The glucose responses were described by AUCi. AUCi (incremental area under the curve) was determined for 120 minutes after the logged meal intakes. The trapezoidal rule was applied in the calculation. The comparison of glycemic response was restricted to participants who completed the glucose test meal in both sensor phases (n=168).

The results showed a lower glycemic response after the supplementation of SAMANA® FORCE (p=0.012).

### Subanalysis of Prediabetic Participants

The previous results raised the question if the glycemic response to the glucose test meal after the intake of SAMANA® FORCE was reduced in all participants or whether people with prediabetes carried the effect. Therefore, the glycemic responses to the glucose test meal were analysed separately for people with (n=57) and without (n=110) prediabetes. The fasting glucose at the beginning of the intervention indicated the presence (>=100 mg/dl) or absence (<100 mg/dl) of a prediabetes. Afterwards the responses were compared between both groups. Only data from participants who completed the glucose test meal in both sensor phases were used for the analyses. The data of one prediabetic participant were removed since the glycemic response indicated that the glucose test meal had been eaten incorrectly (before=13.71 mg/dl, after=125.88 mg/dl).

The analysis of prediabetic participants showed a significant decrease of the glycemic response to the glucose test meal after the supplementation of SAMANA® FORCE (p=0.012). The results are summarized in table 13.

TABLE 13
Reaction to glucose test meal before and after supplementation with
SAMANA ® FORCE In contrast, no significant change in in
glycemic response to a standardized glucose test meal of participants
without prediabetes was found (p = 0.345), as shown in table 14.

| Variable | n | before | after | Diff. | p-value |
|---|---|---|---|---|---|
| AUCi (mg/dl) | 168 | 71.44 (±37.39) | 65.13 (±35.54) | −6.31 | 0.012* |

*Wilcoxon-test

TABLE 14
Glycemic response to a standardized glucose test meal determined
by AUCi of participants with and without prediabetes.

| Variable | n | before | after | p-value | delta (%) |
|---|---|---|---|---|---|
| Prediabetes | 57 | 82.10 | 70.41 | 0.005* | −7.97 |
| | | (±41.52) | (±38.25) | | (±41.07) |
| No Prediabetes | 110 | 66.44 | 61.84 | 0.345* | 4.18 |
| | | (±33.78) | (±33.53) | | (±50.11) |

Mann-Whitney-U-Test

Additionally, the changes (delta %) in glycemic responses over the course of the study were compared between participants with and without prediabetes. Changes in percent did not differ significantly between both groups (p=0.072).

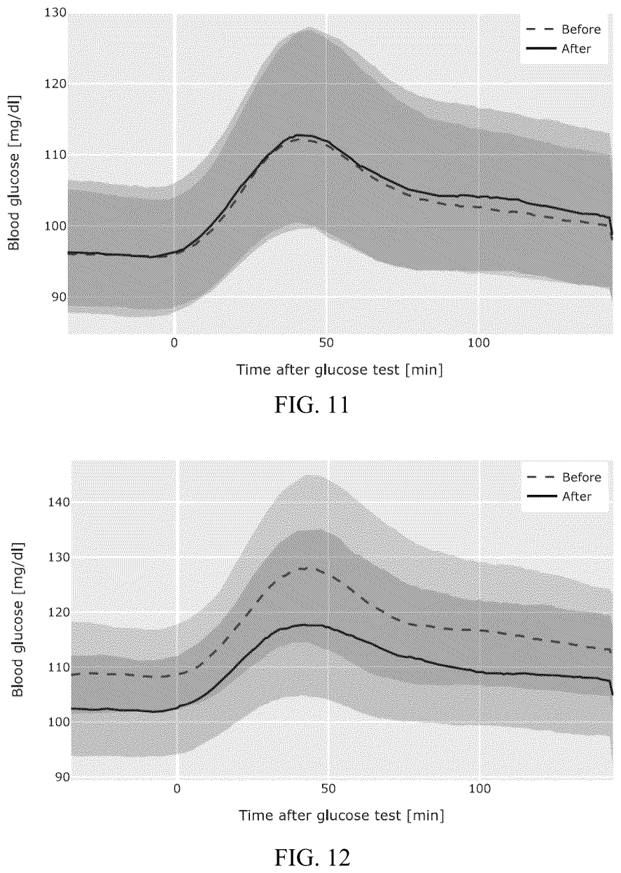

Additionally, the course of the post-prandial glucose level (PPGL) overtime during oral glucose tolerance test (OGTT) were compared from −20 minutes before glucose ingestion to 140 minutes post-prandial. A pronounced reduction on postprandial glucose level could be detected after the supplementation period for all participants as shown in table 15. However, the effect was even stronger for the prediabetic participants as shown in FIGS. 11 and 12.

TABLE 15
Change of blood glucose after the glucose test meal for
all participants (average absolute values in mg/dl)

| | | |
|---|---|---|
| Time in minutes | before | after |

| | | | | |
|---|---|---|---|---|
| −35 | 91.35 | (±16.02) | 92.64 | (±12.26) |
| −20 | 94.63 | (±18.41) | 95.04 | (±13.23) |
| −5 | 99.25 | (±19.19) | 98.28 | (±13.24) |
| 10 | 112.37 | (±20.52) | 111.24 | (±15.16) |
| 25 | 139.61 | (±23.98) | 137.96 | (±25.69) |
| 40 | 154.20 | (±27.45) | 150.93 | (±34.75) |
| 55 | 147.04 | (±34.32) | 144.59 | (±38.78) |
| 70 | 132.31 | (±36.95) | 131.06 | (±37.77) |
| 85 | 117.65 | (±29.95) | 117.41 | (±32.43) |
| 100 | 107.85 | (±24.12) | 106.97 | (±26.49) |
| 115 | 100.29 | (±20.04) | 97.85 | (±21.50) |
| 130 | 94.17 | (±18.43) | 91.11 | (±18.17) |

### Analysis of HbA1c Values and Average Glucose Level

HbA1c was calculated by multiplying the average glucose level of the complete test phase with 0.03 and adding 2.6. The results are summarized in table 16. Average glucose levels were determined by continuous glucose monitoring and are summarized in table 17.

TABLE 16
HbA1c values before and after supplementation
with SAMANA ® FORCE

| Group of persons | before | after | p-value (paired t-test) |
|---|---|---|---|
| Whole study group | 5.72% | 5.65% | <0.001 |
| prediabetes | 6.01% | 5.81% | <0.001 |
| No prediabetes | 5.58% | 5.57% | 0.767 |

TABLE 17
Average (not fasting) glucose level (in mg/dl) before and
after supplementation with SAMANA ® FORCE

| Group of persons | before | after | p-value (paired t-test) |
|---|---|---|---|
| Whole study group | 104.14 | 101.81 | <0.001 |
| prediabetes | 113.62 | 106.98 | <0.001 |
| No prediabetes | 99.34 | 99.10 | 0.750 |

### Analysis of Butyrate Production in In Vitro Gut Models

### Example 6: Butyrate Production of Different Combinations of Bacterial Strains and Dipeptides

### Intestinal Screening Model

To determine the effect of the probiotic strain Bacillus subtilis DSM 32315 on adult colonic microbiota, an intestinal screening model was used (i-screen, TNO, the Netherlands). Therefore the i-screen model was inoculated with standard human adult fecal microbiota material, which consisted of pooled fecal donations from 6 healthy adult volunteers (Caucasian, European lifestyle and nutrition). The fecal material was mixed and grown in a fed-batch fermenter for 40 hours to create a standardized microbiota as described previously [23]. These standard adult gut microbiota sets were stored at −80° C. in 12% glycerol.

The intestinal microbiota was cultured in vitro in modified standard ileal efflux medium (SIEM) with a modified composition: 0.047 g/l pectin, 0.047 g/l xylan, 0.047 g/l arabinogalactan, 0.047 g/I amylopectin, 0.392 g/l starch, 24.0 g/l casein, 24.0 Bacto pepton, 0.4 ox-bile and 0.2 g/l cysteine.

All components were supplied by Trititium Microbiology (Veldhoven, The Netherlands). The pH of the medium was adjusted to 5.8.

For the i-screen fermentations, the precultured standardized fecal inoculum was diluted 50 times in 1350 μl modified SIEM. All experiments have been carried out in triplicates. The strains Bacillus subtilis (DSM 32315) and others were precultured separately in 50 ml LBKelly medium, for about 16 h. Incubation was done in shaking flasks at 37° C. under aerobic conditions. After incubation, bacterial density was determined by optical density measurement at 600 nm. A final stock solution of 1×10¹⁰ cells/ml was prepared in 1 ml buffer solution (0.1 mM MES pH 6). The suspension of each strain was introduced into the i-screen to a final level of about 10⁹ cells/ml, respectively The i-screen incubation was performed under following gas conditions: 0.2% O₂, 0.2% CO₂, 10% H₂, 89.6% N₂.

The probiotic strains Bacillus subtilis DSM 32315 and further control strains (Bacillus strain B and Bacillus strain C) do not produce detectable levels of n-butyrate after exposure in SIEM for 24 h, but they have significant positive influences (p-values<0.05) on the level of n-butyrate production by the human microbiota in combination with the dipeptide Ala-Gln. The results are summarized in FIG. 13.

FIG. 13 shows after 24 h incubation in SIEM measured n-butyrate concentrations in mM in the presence of colon microbiota containing Bacillus subtilis DSM 32315 or control strains Bacillus strain B or Bacillus strain C in combination with the dipeptide Ala-Gln.

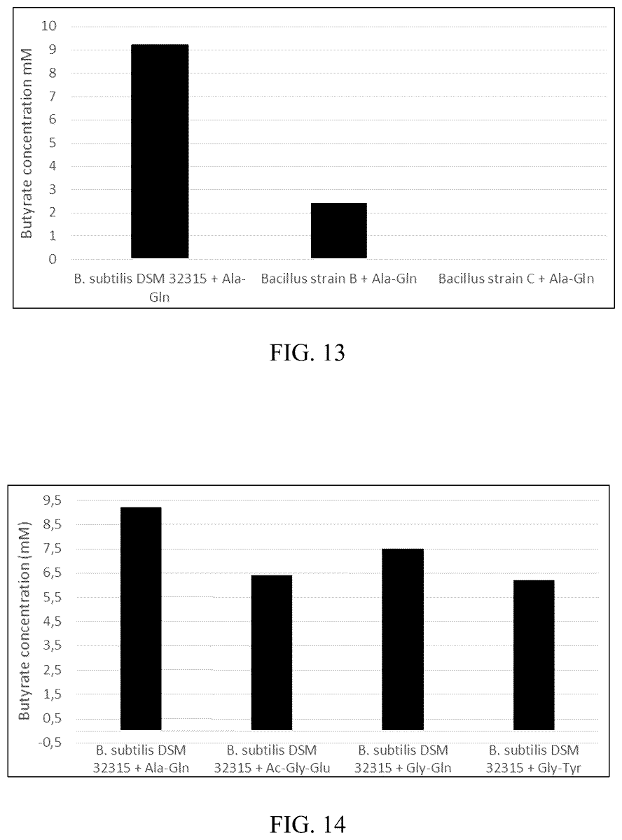

A comparison of combination products of the best B. subtilis strain DSM 32315 with different dipeptides showed that the combination product with the dipeptide Ala-Gln had the best effect on butyrate production. The results are summarized in FIG. 14.

FIG. 14 shows after 24 h incubation in SIEM measured n-butyrate concentrations in mM in the presence of colon microbiota containing Bacillus subtilis DSM 32315 in combination with different dipeptides Ala-Gln, Ac-Gly-Glu, Gly-Gln and Gly-Tyr.

### Example 7: Butyrate Production for Synbiotic Products with Different Coating Compositions

Moreover, the effect of different coating compositions on butyrate production were analyzed. The colonic coating comprises EUDRAGUARD® biotic, whereas the enteric coating comprises EUDRAGUARD® natural.

EUDRAGUARD® biotic can start to disintegrate at pH conditions in the lower small intestine and the colon (pH>7.0). The coating comprises a polymer composition, wherein the polymer is polymerized from 20 to 30% by weight methyl methacrylate, 60 to 70% by weight methyl acrylate and 8 to 12% by weight methacrylic acid. It is further preferred, when the polymer is polymerized from 25% by weight methyl methacrylate, 65% by weight methyl acrylate and 10% by weight methacrylic acid.

EUDRAGUARD® natural is a maize starch-based coating comprising modified starch.

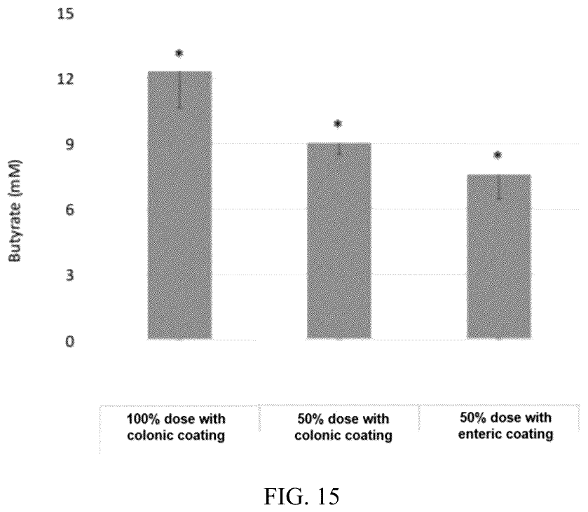

The results on butyrate production are summarized in table 18 and FIG. 15.

TABLE 18
Butyrate production in in vitro gut model with different
coating compositions using 2 capsules daily dose (100%
dose) or 1 capsule daily dose (50% dose)

| | 100% dose | 50% dose | 50% dose |
|---|---|---|---|
| | (colonic | (colonic | (enteric |
| | coating) | coating) | coating) |
| 24-48 hours; Butyrate (mM) | 12.27 | 8.93 | 7.5 |
| 16-24 hours; Butyrate (mM) | 0.03 | 0.04 | 0.05 |
| 3-16 hours; Butyrate (mM) | 0.00 | 0.01 | 0.01 |

FIG. 15 shows butyrate production in in vitro gut model with different coating compositions using 2 capsules daily dose (100% dose) or 1 capsule daily dose (50% dose). *=significantly different from control

100% daily dose (=2 capsules) contains:

Bacillus subtilis DSM 32315 spore powder (˜with 2 billion CFU), L-Alanyl-L-Glutamine (290 mg), 90 mg extract of Curcuma, 90 mg extract of green tea, D- and B-vitamin(s) and mineral.

### REFERENCES

1. Gasaly N, Hermoso M A, Gotteland M: Butyrate and the Fine-Tuning of Colonic Homeostasis: Implication for Inflammatory Bowel Diseases. Int J Mol Sci 2021, 22(6).
2. Boets E, Gomand S V, Deroover L, Preston T, Vermeulen K, De Preter V, Hamer H M, Van den Mooter G, De Vuyst L, Courtin C M et al: Systemic availability and metabolism of colonic-derived short-chain fatty acids in healthy subjects: a stable isotope study. J Physiol 2017, 595(2):541-555.
3. Nogal A, Valdes A M, Menni C: The role of short-chain fatty acids in the interplay between gut microbiota and diet in cardio-metabolic health. Gut Microbes 2021, 13(1):1-24.
4. Salamone D, Rivellese A A, Vetrani C: The relationship between gut microbiota, short-chain fatty acids and type 2 diabetes mellitus: the possible role of dietary fibre. Acta Diabetol 2021.
5. Hou J K, Abraham B, El-Serag H: Dietary intake and risk of developing inflammatory bowel disease: a systematic review of the literature. Am J Gastroenterol 2011, 106(4):563-573.
6. Vernia P, Annese V, Bresci G, d'Albasio G, D'Inca R, Giaccari S, Ingrosso M, Mansi C, Riegler G, Valpiani D et al: Topical butyrate improves efficacy of 5-ASA in refractory distal ulcerative colitis: results of a multicentre trial. Eur J Clin Invest 2003, 33(3):244-248.
7. Roda A, Simoni P, Magliulo M, Nanni P, Baraldini M, Roda G, Roda E: A new oral formulation for the release of sodium butyrate in the ileo-cecal region and colon. World J Gastroenterol 2007, 13(7):1079-1084.
8. Kaminski M, Skonieczna-Zydecka K, Nowak J K, Stachowska E: Global and local diet popularity rankings, their secular trends, and seasonal variation in Google Trends data. Nutrition 2020, 79-80:110759.
9. Halmos T, Suba I: [Physiological patterns of intestinal microbiota. The role of dysbacteriosis in obesity, insulin resistance, diabetes and metabolic syndrome]. Orv Hetil 2016, 157(1):13-22.
10. Boucher J, Kleinridders A, Kahn C R: Insulin receptor signaling in normal and insulin-resistant states. Cold Spring Harb Perspect Biol 2014, 6(1).
11. Vital M, Karch A, Pieper D H: Colonic Butyrate-Producing Communities in Humans: an Overview Using Omics Data. mSystems 2017, 2(6).
12. Perraudeau F, McMurdie P, Bullard J, Cheng A, Cutcliffe C, Deo A, Eid J, Gines J, Iyer M, Justice N et al: Improvements to postprandial glucose control in subjects with type 2 diabetes: a multicenter, double blind, randomized placebo-controlled trial of a novel probiotic formulation. BMJ Open Diabetes Res Care 2020, 8(1).
13. Stoeva M K, Garcia-So J, Justice N, Myers J, Tyagi S, Nemchek M, McMurdie P J, Kolterman O, Eid J: Butyrate-producing human gut symbiont, Clostridium butyricum, and its role in health and disease. Gut Microbes 2021, 13(1):1-28.
14. Boesmans L, Valles-Colomer M, Wang J, Eeckhaut V, Falony G, Ducatelle R, Van Immerseel F, Raes J, Verbeke K: Butyrate Producers as Potential Next-Generation Probiotics: Safety Assessment of the Administration of Butyricicoccus pullicaecorum to Healthy Volunteers. mSystems 2018, 3(6).
15. Reidelberger R, Haver A, Anders K, Apenteng B, Lanio C: Effects of solid-phase extraction of plasma in measuring gut metabolic hormones in fasted and fed blood of lean and diet-induced obese rats. Physiol Rep 2016, 4(10).
16. de Luis D A, Gonzalez Sagrado M, Conde R, Aller R, Izaola O: Decreased basal levels of glucagon-like peptide-1 after weight loss in obese subjects. Ann Nutr Metab 2007, 51(2):134-138.
17. Adam T C, Jocken J, Westerterp-Plantenga M S: Decreased glucagon-like peptide 1 release after weight loss in overweight/obese subjects. Obes Res 2005, 13(4):710-716.
18. Ranganath L, Norris F, Morgan L, Wright J, Marks V: Inhibition of carbohydrate-mediated glucagon-like peptide-1 (7-36)amide secretion by circulating non-esterified fatty acids. Clin Sci (Lond) 1999, 96(4):335-342.
19. Verdich C, Toubro S, Buemann B, Lysgard Madsen J, Juul Holst J, Astrup A: The role of postprandial releases of insulin and incretin hormones in meal-induced satiety—effect of obesity and weight reduction. Int J Obes Relat Metab Disord 2001, 25(8):1206-1214.
20. Wu Y, Zhang Q, Ren Y, Ruan Z: Effect of probiotic Lactobacillus on lipid profile: A systematic review and meta-analysis of randomized, controlled trials. PLoS One 2017, 12(6):e0178868.
21. Qin P, Zou Y, Dai Y, Luo G, Zhang X, Xiao L: Characterization a Novel Butyric Acid-Producing Bacterium Collinsella aerofaciens Subsp. Shenzhenensis Subsp. Nov. Microorganisms 2019, 7(3).
22. Millan J, Pinto X, Munoz A, Zuniga M, Rubies-Prat J, Pallardo L F, Masana L, Mangas A, Hernandez-Mijares A, Gonzalez-Santos P et al: Lipoprotein ratios: Physiological significance and clinical usefulness in cardiovascular prevention. Vasc Health Risk Manag 2009, 5:757-765.
23. Ladirat S E, Schols H A, Nauta A, Schoterman M H, Keijser B J, Montijn R C, Gruppen H, Schuren F H: High-throughput analysis of the impact of antibiotics on the human intestinal microbiota composition. J Microbiol Methods 2013, 92(3):387-397.
24. Faerch, K.; Torekov, S. S.; Vistisen, D.; Johansen, N. B.; Witte, D. R.; Jonsson, A.; Pedersen, O.; Hansen, T.; Lauritzen, T.; Sandbaek, A.; et al. GLP-1 Response to Oral Glucose Is Reduced in Prediabetes, Screen-Detected Type 2 Diabetes, and Obesity and Influenced by Sex: The ADDITION-PRO Study. Diabetes 2015, 64, 2513-2525. [CrossRef]
25. Seon, M. J.; Hwang, S. Y.; Son, Y.; Song, J.; Kim, O. Y. Circulating GLP-1 Levels as a Potential Indicator of Metabolic Syndrome Risk in Adult Women. Nutrients 2021, 13, 865. [CrossRef]
26. Adam, T. C.; Jocken, J.; Westerterp-Plantenga, M. S. Decreased glucagon-like peptide 1 release after weight loss in overweight/obese subjects. Obes. Res. 2005, 13, 710-716. [CrossRef]
27. De Luis, D. A.; Gonzalez Sagrado, M.; Conde, R.; Aller, R.; Izaola, O. Decreased basal levels of glucagon-like peptide-1 after weight loss in obese subjects. Ann. Nutr. Metab. 2007, 51, 134-138. [CrossRef]

FIG. 1: Distribution of fasting blood glucose levels [mg/dl]; scatter plot with mean±95% Cl

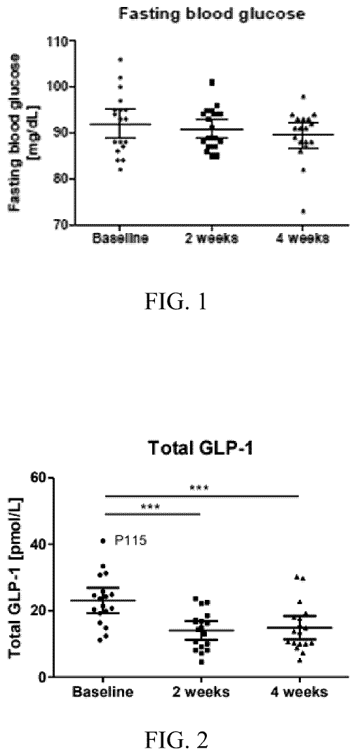

FIG. 2: Distribution of Total GLP-1 levels [pmol/L]; scatter plot with mean±95% Cl; Baseline vs. 2 weeks p<0.001 (paired t-test); Baseline vs. 4 weeks p<0.001 (paired t-test)

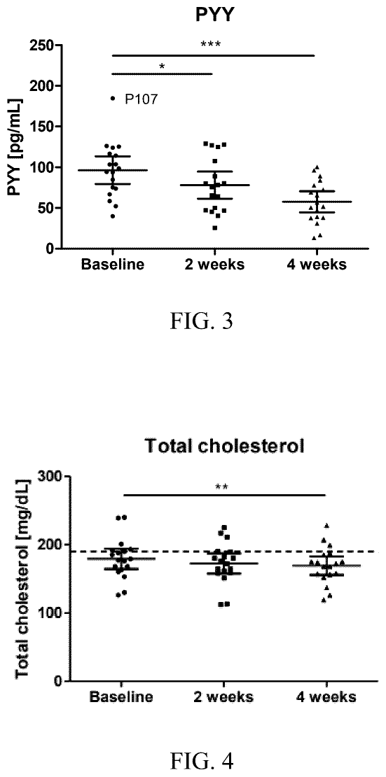

FIG. 3: Distribution of PYY levels [pg/mL]; scatter plot with mean±95% Cl; Baseline vs. 2 weeks p=0.0139 (paired t-test); Baseline vs. 4 weeks p<0.001 (paired t-test)

FIG. 4: Distribution of total cholesterol levels [mg/dL]; scatter plot with mean±95% Cl; Baseline vs. 4 weeks p=0.0037 (paired t-test)

FIG. 5: Distribution of HDL cholesterol levels [mg/dL]; scatter plot with mean±95% Cl

FIG. 6: Distribution of triglyceride levels [mg/dL]; scatter plot with mean±95% Cl

FIG. 7: Distribution of LDL/HDL cholesterol ratio; scatter plot with mean±95% Cl; Baseline vs. 4 weeks p=0.0022 (paired t-test)

FIG. 8: Collinsella aerofaciens [relative abundance] in stool samples by time point; scatter plot with mean±95% Cl; Baseline vs. 4 weeks p=0.0179 (Wilcoxon signed rank test)

FIG. 9: Fasting glucose before and after supplementation of SAMANA® FORCE. Paired t test was used for analysis. Statistics are shown.

FIG. 10: Correlation between fasting glucose at baseline and change (delta %) of fasting glucose. Spearman correlation was used for analysis. Statistics are shown.

FIG. 11: PPGR of glucose test meal before and after intervention of non-prediabetic participants (Median and IQR)

FIG. 12: PPGR of glucose test meal before and after intervention of prediabetic participants (Median and IQR)

FIG. 13: Butyrate production in in vitro gut model by synbiotic combinations containing different combinations of B. subtilis strains and dipeptides

FIG. 14: Butyrate production in in vitro gut model by combinations of B. subtilis DSM 32315 with different dipeptides

FIG. 15: Butyrate production in in vitro gut model with different coating compositions using 2 capsules daily dose (100% dose) or 1 capsule daily dose (50% dose), *=significantly different from control

1. A preparation for modulating satiety hormone levels in a subject, wherein the preparation comprises,
at least one probiotic Bacillus subtilis strain, and
at least one dipeptide comprising a glutamine or glutamic acid unit, and wherein the satiety hormone is selected from the group consisting of CCK, GLP-1 and PYY.

2. The preparation according to claim 1, wherein the preparation is to be administered to the subject via at least 1 oral dosage of at least 1 billion CFU of the probiotic Bacillus subtilis strain and at least 250 mg dipeptide per day.

3. The preparation according to claim 2, wherein the modulation of satiety hormones is a reduction of satiety hormone level of at least 5% after 4 weeks of administration of the preparation.

4. The preparation according to claim 1, further comprising one or more plant extracts selected from a curcuma extract and green tea extract.

5. The preparation according to claim 1, wherein the probiotic strain is selected from the group consisting of Bacillus subtilis DSM 32315, Bacillus subtilis DSM 32540, and Bacillus subtilis DSM 32592.

6. The preparation according to claim 1, wherein the at least one dipeptide is selected from the group consisting of glycine-glutamine, glycine-glutamic acid, alanine-glutamine, alanine-glutamic acid and its acetylated forms.

7. The preparation according to claim 1, wherein a total amount of probiotic strain and amino acid or oligopeptide is at least 40 weight-% of a total weight of the preparation.

8. The preparation according to claim 4, wherein a total amount of plant extracts is at least 10 weight-% of the total weight of the preparation.

9. The preparation according to claim 1, wherein the preparation comprises an enteric coating, wherein the enteric coating comprises at least one selected from the group consisting of methyl acrylate-methacrylic acid copolymers, cellulose acetate phthalate (CAP), cellulose acetate succinate, hydroxypropyl methyl cellulose phthalate, hydroxypropyl methyl cellulose acetate succinate (hypromellose acetate succinate), polyvinyl acetate phthalate (PVAP), methyl methacrylate-methacrylic acid copolymers, shellac, cellulose acetate trimellitate, sodium alginate, and zein.

10. The preparation according to claim 1, wherein the preparation comprises an enteric coating comprising a polymer composition, wherein the polymer composition is polymerized from 20 to 30% by weight methyl methacrylate, 60 to 70% by weight methyl acrylate and 8 to 12% by weight methacrylic acid.

11. A method of preventing and treating obesity, adiposity, type 2 diabetes and metabolic syndrome, the method comprising:
administering the preparation according to claim 1 to a subject in need thereof.

12. A preparation for decreasing fasting glucose in a subject, wherein the preparation comprises:
at least one probiotic Bacillus subtillis strain, and
at least one dipeptide comprising a glutamine or glutamic acid unit.

13. The preparation according to claim 12, wherein the probiotic Bacillus subtilis strain is Bacillus subtilis DSM 32315 and the at least one dipeptide is alanine-glutamine.

14. The preparation according to claim 12, wherein a glycemic response in the subject is reduced.

15. The preparation according to claim 12, wherein the subject is human and in a prediabetic state, having a fasting glucose value of more than 100 mg/dl.

16. The preparation according to claim 15, wherein a body weight of the subject is reduced by at least 1 kg after 4 weeks of administration of the preparation.

17. The preparation according to claim 2, wherein the preparation is to be administered to the subject via at least 2 oral dosages per day.

18. The preparation according to claim 3, wherein the modulation of satiety hormones is a reduction of satiety hormone level of at least 30 after 4 weeks of administration of the preparation.

19. The preparation according to claim 5, wherein the probiotic strain is Bacillus subtilis DSM 32315.

20. The preparation according to claim 7, wherein the total amount of probiotic strain and amino acid or oligopeptide is at least 70 weight-% of the total weight of the preparation.
